(19) United States
(12) Patent Application Publication
(10) Pub. No.: US 2007/0010848 A1
(43) Pub. Date: Jan. 11, 2007

(54) Systems and methods for providing cavities in interior body regions

(76) Inventors: Andrea Leung, Milpitas, CA (US); Cesar Ico, San Francisco, CA (US); Alberto Cantu, San Francisco, CA (US)

Correspondence Address: KILPATRICK STOCKTON LLP, 1001 WEST FOURTH STREET, WINSTON-SALEM, NC, 27101, US

(21) Appl. No.: 11/178,662

(22) Filed: Jul. 11, 2005

(51) Int. Cl. A61M 29/00

(52) U.S. Cl. 606/198

(57) ABSTRACT

Systems and methods for providing a cavity in an interior body region are described. In one described method, a spring disposed between a first plate and a second plate is compressed. The compressed spring is inserted into a cannula comprising a cannula distal end. The spring is decompressed, at least in part, once at least one of the first and second plates is inserted beyond the cannula distal end to a treatment area adjacent a tissue. The decompression of the spring increases a distance between the first and second plates.

### FIELD OF THE INVENTION

The invention relates to systems and methods for providing cavities in interior body regions for diagnostic or therapeutic purposes.

### BACKGROUND

Certain diagnostic or therapeutic procedures require provision of a cavity in an interior body region. For example, as disclosed in U.S. Pat. Nos. 4,969,888 and 5,108,404, a balloon may be deployed to form a cavity in cancellous bone tissue, as part of a therapeutic procedure that fixes fractures or other abnormal bone conditions, both osteoporotic and non-osteoporotic in origin. The balloon or other expansible body may compress the cancellous bone to form an interior cavity. The cavity may receive a filling material, such as a bone cement, which provides renewed interior structural support for cortical bone.

This procedure can be used to treat cortical bone, which due to osteoporosis, avascular necrosis, cancer, trauma, or other disease is fractured or is prone to compression fracture or collapse. These conditions, if not successfully treated, can result in deformities, chronic complications, and an overall adverse impact upon the quality of life. However, as a balloon is inflated during such a procedure, it may not expand to a shape and dimension desired by a user of the device, may place too much pressure on a bone tissue weakened by an osteoporotic condition, or may be unable to provide sufficient force to break apart a healed bone fracture.

A demand exists for further systems and methods that are capable of providing cavities in bone and other interior body regions in safe and efficacious ways.

### SUMMARY

Embodiments of the present invention provide systems and methods for providing cavities in interior body regions. One illustrative embodiment comprises a cannula comprising a cannula distal end, an elongate member comprising an elongate member distal end, a first plate, a second plate, and a spring disposed between the first and second plates. Both the first and second plates may be configured to be slidably disposed within the cannula and coupled to the elongate member distal end. The spring may be configured to be at least partly compressed when the first and second plates are within the cannula, and to decompress, at least in part, as at least one of the first and second plates is extended beyond the cannula distal end. The decompression of the spring may increase a distance between the first and second plates.

This embodiment is mentioned not to limit or define the invention, but to provide an example of an embodiment of the invention to aid understanding thereof. Illustrative embodiments are discussed in the Detailed Description, and further description of the invention is provided there. Advantages offered by the various embodiments of the present invention may be further understood by examining this specification.

### BRIEF DESCRIPTION OF THE FIGURES

These and other features, aspects, and advantages of the present invention are better understood when the following Detailed Description is read with reference to the accompanying drawings, wherein:

FIG. 1 is an elevation view of a tool according to one embodiment of the present invention;

FIG. 2 is a cut-away elevation view of the tool shown in FIG. 1, wherein the first and second plates are shown within the cannula;

FIG. 3 is an elevation view of the tool shown in FIGS. 1 and 2, wherein the first and second plates are shown partially retracted into the cannula;

FIG. 4 is an elevation view of a tool according to another embodiment of the present invention;

FIG. 5 is an elevation (lateral) view of several human vertebrae, with a cannula establishing a percutaneous path to a vertebral body of one of the several vertebrae;

FIG. 6 is an elevation (lateral) view of a human vertebra comprising a vertical compression fracture condition and with a tool according to one embodiment of the present invention deployed to enlarge a cavity within a vertebral body;

FIG. 7 is an elevation (lateral) view of the tool and vertebra of FIG. 6, wherein the tool is shown extended beyond the distal end of the cannula, and the cavity within the vertebral body is shown with an increased internal dimension resulting from insertion of the tool;

FIG. 8 is a flow chart of a method according to one embodiment of the present invention;

FIG. 9 is a plan view of a sterile kit configured to store a single use tool according to one embodiment of the present invention; and

FIG. 10 is an exploded perspective view of the sterile kit of FIG. 9.

### DETAILED DESCRIPTION

Embodiments of the present invention provide systems and methods for providing cavities in interior body regions. The systems and methods embodying the invention can be adapted for use in many suitable interior body regions, wherever the formation or enlargement of a cavity within or adjacent one or more layers of tissue may be required for a therapeutic or diagnostic purpose. The illustrative embodiments show the invention in association with systems and methods used to treat bones. In other embodiments, the present invention may be used in other interior body regions or types of tissues.

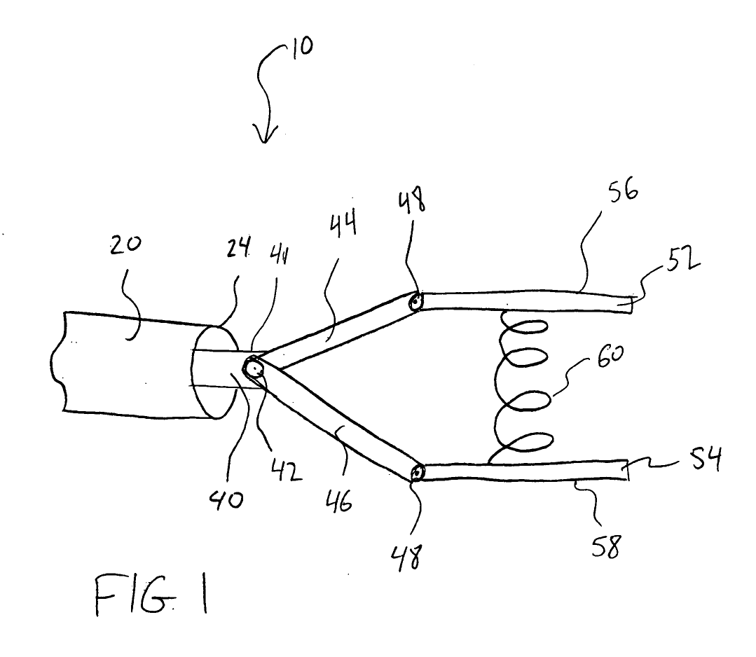

Referring now to the Figures, in which like part numbers depict like elements throughout the Figures, FIG. 1 is an elevation view of a system 10 according to one embodiment of the present invention. The system 10 shown in FIG. 1 is configured to allow an operator to provide a cavity in a targeted treatment area. The system 10 is further configured to be used in a kyphoplasty procedure to restore height to a vertebra suffering from a vertical compression fracture condition.

The system 10 comprises a cannula 20 comprising a proximal end (not shown) and a distal end 24. The cannula 20 may be fabricated from a material selected to facilitate advancement and rotation of an elongate member 40 movably disposed within the cannula 20. The cannula 20 can be constructed, for example, using standard flexible, medical grade plastic materials, such as vinyl, nylon, polyethylenes, ionomer, polyurethane, and polyethylene tetraphthalate (PET). At least some portion of the cannula 20 can also comprise more rigid materials to impart greater stiffness and thereby aid in its manipulation and torque transmission capabilities. More rigid materials that can be used for this purpose comprise stainless steel, nickel-titanium alloys (such as Nitinol), and other metal alloys.

The system 10 shown in FIG. 1 further comprises the elongate member 40 movably disposed within the cannula 20. The elongate member 40 may be made from a resilient inert material providing torsion transmission capabilities (e.g., stainless steel, a nickel-titanium alloy such as Nitinol, and other suitable metal alloys). In other embodiments, the elongate member 40 may be fashioned from a variety of suitable materials, comprising a carbon fiber, a glass, or a flexible material, such as a plastic or rubber. In one embodiment comprising a flexible elongate member 40, the elongate member 40 may be, for example, fashioned from twisted wire filaments, such stainless steel, nickel-titanium alloys (such as Nitinol), and suitable other metal alloys.

The elongate member 40 shown comprises a hinge 42 at its distal end 41. The elongate member 40 may comprise a handle (not shown) at its proximal end (not shown) to aid in gripping and maneuvering the elongate member 40. For example, in one embodiment, such a handle can be made of a foam material secured about the proximal end elongate member 40.

The system 10 further comprises a first coupling arm 44 and a second coupling arm 46. The first and second coupling arms 44, 46 in the embodiment shown in FIGS. 1-3 are both rotatably coupled to the hinge 42 at their proximal ends. The hinge 42, and the first and second coupling arms 44, 46 are configured to so as to allow the coupling arms 44, 46 to rotate about the hinge 42 independently. In another embodiment, the coupling arms 44, 46 may be coupled to the elongate member 40 in such a manner that when either of the two coupling arms 44, 46 moves with respect to the elongate member 40, the other coupling arm 44, 46 also moves with respect to the elongate member 40. For example, in one such embodiment, the coupling arms 44, 46 may be coupled to each other and the distal end 41 of the elongate member 40 through a gear mechanism.

Both the first and second coupling arms 44, 46 comprise a hinge 48 at their distal ends. Rotatably coupled to the hinge 48 at the distal end of the first coupling arm 44 is a first plate 52. Similarly, rotatably coupled to the hinge 48 at the distal end of the second coupling arm 46 is a second plate 54. In another embodiment of the present invention, one or both of the plates 52, 54 may be coupled directly to the distal end 41 of the elongate member 40. In one such embodiment, the distal end 41 of the elongate member 40 may comprise a first deflectable beam rigidly coupled to the first plate 52, and a second deflectable beam rigidly coupled to the second plate 54. The system 10, as shown in FIG. 1, is in an extended state, wherein the first and second plates 52, 54, and the first and second coupling arms 44, 46 are all extended beyond the distal end 24 of the cannula 20.

The first and second plates 52, 54 may be coupled to the coupling arms 44, 46, respectively, or to the distal end 41 of the elongate member 40 through the use of welding, gluing, bonding, melting, a ball joint, a universal joint, or any other suitable fastener (such as a screw, a rivet, a tack, a staple, a nail, etc.). In yet another embodiment, the elongate member 40 and the first and second plates 52, 54 may be fashioned from the same material, and may be injection molded, cast, forged, or machined as a solid element.

In the embodiment shown in FIGS. 1-3, the first and second coupling arms 44, 46 and the first and second plates 52, 54 are all fashioned from stainless steel. In other embodiments one or more of the coupling arms 44, 46 and the plates 52, 54 may be fashioned from a different suitable resilient inert material, such as a surgical grade shape memory material formed with a resilient memory, such as a nickel titanium alloy. In yet other embodiments, one or more of the coupling arms 44, 46 and the plates 52, 54 may be fashioned from a variety of other suitable materials, comprising a carbon fiber, a glass, or a flexible material, such as a plastic or rubber.

The first plate 52 comprises a first surface 56 facing a first direction. The second plate 54 comprises a second surface 58 facing a second direction. The first and second surfaces 56, 58 are configured to be deployed in a treatment area adjacent body tissue. In the embodiment shown in FIGS. 1-3, the first and second surfaces 56, 58 both are substantially planar. In another embodiment, one or both of the surfaces 56, 58 may comprise another form. For example, in one embodiment, the first surface 56 may be irregular, and be configured to place pressure on one or more predetermined points within a treatment area adjacent a body tissue.

The system 10 further comprises a spring 60 disposed between the first and second plates 52, 54. The spring 60 is shown in FIG. 1 in a decompressed state. Accordingly, in the embodiment described with respect to FIGS. 1-3, the distance between the first and second plates 52, 54 measured parallel to the axis of the spring 60 is greatest when the spring 60 is decompressed as shown in FIG. 1. In the embodiment shown in FIGS. 1-3, the fully expanded distance between the first and second plates 52, 54 is greater than the inside diameter of the interior bore of the cannula 20. In other embodiments, the system 10 may be configured to expand differently, and to a lesser extent. For example, in one such embodiment, the maximum distance between the first and second plates 52, 54 when they are extended beyond the distal end 24 of the cannula 20 may be lesser than or equal to the interior bore of the cannula 20.

The spring 60 in the embodiment shown in FIGS. 1-3 comprises a helical spring fashioned from stainless steel. In other embodiments, other types of spring, additional springs, or springs fashioned from a different material (such as a nickel-titanium alloy) may be disposed between the first and second plates 52, 54. For example, in one embodiment, a plurality of leaf springs may be disposed between the first and second plates 52, 54. In yet another embodiment, one or more deflectable beams fashioned form a shape memory material (such as Nitinol) may be disposed between the first and second plates 52, 54 either in addition to the spring 60, or in its place.

In the embodiment shown in FIGS. 1-3, the first and second plates 52, 54 are substantially parallel at all times during operation. In another embodiment, the first and second plates 52, 54 may be substantially parallel only when the spring 60 is at least partly decompressed. For example, in one such embodiment, the first and second plates 52, 54 may be parallel only when both the first and second plates 52, 54 are extended beyond the distal end 24 of the cannula 20.

In the embodiment shown, the first and second directions that the first surface 56 and the second surface 58 face, respectively, are separated by 180 degrees during all times of operation. In another embodiment, the first and second surfaces 56, 58 may face first and second directions, respectively, that are separated by greater or fewer than 180 degrees, or may be separated by 180 degrees only while the spring 60 is at least partly decompressed.

In another embodiment, one or both of the plates 52, 54 may comprise at least one sharp edge configured to contact and shear (curette) tissue in a treatment area. In one such embodiment, while the first and second plates 52, 54 are extended beyond the distal end 24 of the cannula 20, the elongate member 40 may be rotated within the cannula 20, thereby rotating the first and second plates 52, 54 within an interior body cavity. In yet another embodiment, another portion of the system 10, such as the coupling arms 44, 46, or the elongate member 40 may comprise a sharp surface configured to directly contact and shear at least one layer of tissue in a treatment area. In another such embodiment, the proximal end of at least one of the cannula 20 and the elongate member 40 may carry a fitting (not shown) that, in use, may be coupled to an electric motor (not shown). The motor may thus rotate one or both of the elongate member 40 and the cannula 20, curetting tissue with the sharp edge.

In one embodiment of the present invention, at least a portion of at least one of the elongate member 40, the first coupling arm 44, the second coupling arm 46, the first plate 52, the second plate 54, and the spring 60 may comprise one or more radiological markers. For example, in the embodiment shown in FIGS. 1-3, the first and second surfaces 56, 58 may each comprise one or more radiological markers. The markers may be fashioned from a radiopaque material, such as platinum, gold, calcium, tantalum, and other heavy metals.

In an embodiment employing a plurality of radiological markers, a first set of markers may be placed at or near a distal end of the plates 52, 54, while another set of markers may be placed at a location on the plates 52, 54 spaced apart from the first set of markers, such as at a point at or near the proximal end of each plate 52, 54. In another embodiment, the distal end 41 of the elongate member 40, or the distal end 24 of the cannula 20 can carry one or more markers. A radiological marker may permit radiologic visualization of at least one of the elements of the system 10 within a targeted treatment area.

A tool according to one embodiment of the present invention, such as the system 10 described with respect to FIGS. 1-3, can comprise an interior lumen. The lumen may be coupled to an external source of fluid and an external vacuum source. In one such embodiment, a rinsing liquid, e.g., sterile saline, can be introduced from the source through the lumen into the targeted tissue region before, during or after the system 10 provides or enlarges a cavity in a tissue mass. The rinsing liquid may reduce friction, conduct heat and bone fragments away from the tissue during an operation. The rinsing liquid can be introduced continuously or intermittently while the tissue mass is being fractured, compacted, removed, or cut. The rinsing liquid can also carry an anticoagulant or other anti-clotting agent. In one such embodiment, the lumen may be coupled to the vacuum source, and liquids and debris can be aspirated from the targeted tissue region through the lumen.

In yet another embodiment of the present invention, a sheath may circumscribe the first and second plates 52, 54. In such an embodiment, the sheath may be disposed between the first and second surfaces 56, 58 and a tissue in a treatment area. Such a sheath may be fabricated from a substantially non-compliant and rupture-resistant material, such as Mylar or a suitable plastic. In a different embodiment, a sheath may comprise a compliant material, such as latex. A sheath according to such an embodiment may prevent dislodged tissue mass from becoming trapped between the first and second plates 52, 54, or within the spring 60.

In another embodiment comprising a sheath, the sheath may comprise an inflatable balloon sheath. The system 10 may thus be used according to the embodiment described with respect to FIGS. 1-3, while also inflating the sheath to increase or alter one or more dimensions of a cavity provided thereby. Such an inflatable balloon sheath may be configured to be deployed adjacent a tissue in a targeted treatment area via the cannula 20.

An inflatable balloon sheath may be disposed at the distal end 41 of the elongate member 40, and circumscribing the first and second plates 52, 54. In one such embodiment of the present invention, the balloon sheath may be configured to be deployed within cancellous bone tissue within a vertebral body through a percutaneous path established by the cannula 20. Such a balloon sheath may comprise a single aperture that is coupled to the distal end 41 of the elongate member 40. The balloon sheath may be inflated by movement of a liquid or a gas through a hollow elongate member 40 and the aperture into the interior of the balloon sheath. The balloon sheath may be deflated by movement of a liquid or a gas out of the balloon sheath through the aperture and a bore through a hollow elongate member 40.

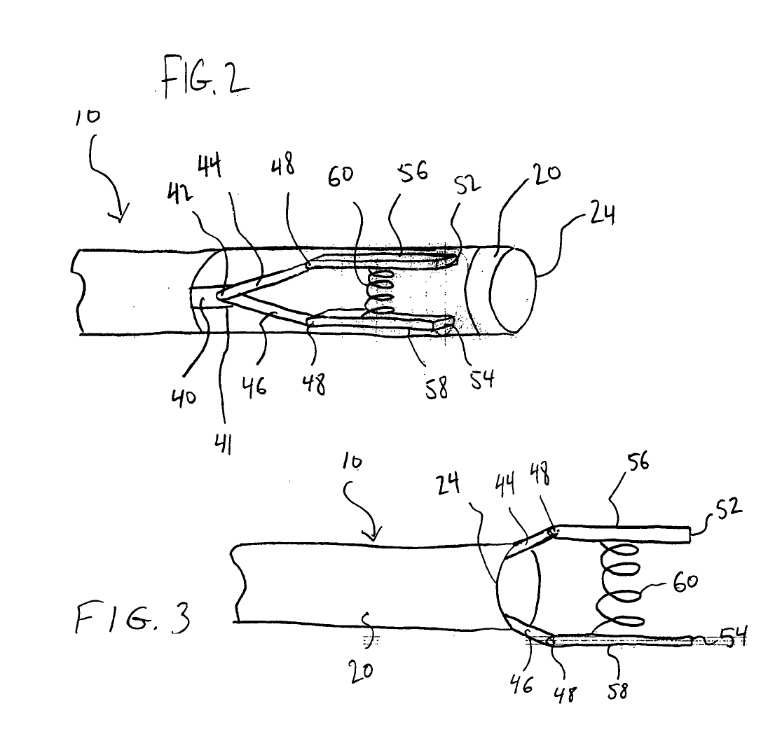

Referring now to FIG. 2, an elevation view of the system 10 shown in FIG. 1, wherein a portion of the cannula 20 has been removed to show the first and second plates 52, 54 therewithin. The system 10, as shown in FIG. 2, is in a retracted state, wherein the first and second plates 52, 54 and the first and second coupling arms 44, 46 are all within the cannula 20. The spring 60, as shown in FIG. 2, has been compressed enough to cause the distance between the first and second plates 52, 54 as measured parallel to the axis of the spring 60 to be less than or equal to the interior bore dimension of the cannula 20.

As shown in FIG. 2, the first and second plates 52, 54 are configured to fit within the cannula 20 when the spring 60 is compressed. In the embodiment shown in FIGS. 1-3, a user of the system 10 may maneuver the first and second plates 52, 54 and the elongate member 40 within and along the axis of the cannula 20 while the spring 60 is compressed, as shown in FIG. 2. In another embodiment, the first and second plates 52, 54, and the elongate member 40 may be configured to be rotated with respect to the cannula 20.

Referring now to FIG. 3, an elevation view of the system 10 is shown, wherein the first and second coupling arms 44, 46 are shown partially within the distal end 24 of the cannula 20. As shown in FIG. 3, the system 10 is in an intermediate state (between the extended state shown in FIG. 1 and the retracted state shown in FIG. 2). While in the system 10 is in the intermediate state, the spring 60 is partially decompressed.

Starting in the intermediate state shown in FIG. 3, the spring 60 may be further compressed as the elongate member 40 is pulled toward the proximal end (not shown) of the cannula 20, thereby drawing the first and second coupling arms 44, 46 further within the distal end 24 of the cannula 20. Conversely, by pushing the elongate member 40 toward the distal end 24 of the cannula 20, the spring 60 may be further decompressed as a greater portion of the first and second coupling arms 44, 46 is moved beyond the distal end 24 of the cannula 20.

In another embodiment, the system 10 may be adjustable. For example, the system 10 may comprise a controller that a user may use to adjust the distance between the first and second plates 52, 54. Using such an embodiment, an operator of the system 10 may use a controller to alter the size and shape of a cavity provided by the system 10.

For example, at least the first and second plates 52, 54 may be in communication with a suitable type of controller, such as a slide controller, a pistol grip controller, a ratcheting controller, or a threaded controller, that can be configured to permit an operator of the system 10 to control at least one of the extent to which the first and second plates 52, 54 extend beyond the distal end 24 of the cannula 20, and the extent to which the distance between the first and second plates 52, 54 is varied. In one such embodiment of the present invention, a controller can also comprise indicia by which an operator can visually estimate the extent to which the distance between the first and second plates 52, 54 has been varied.

In one embodiment comprising a controller, a screw member (not shown) may be coupled to the first and second plates 52, 54 and in communication with a handle (not shown) at the proximal end of the elongate member 40. The screw member may be configured to increase or decrease the distance between the first and second plates 52, 54. By turning the handle, a user of the system 10 may be able to decrease the distance between the first and second plates 52, 54 prior to removing them along with the spring 60 from a treatment area through the cannula 20. In a different embodiment, a screw member may be configured to allow an operator of the system 10 to control compression or decompression of the spring 60, and thereby the distance between the first and second plates 52, 54.

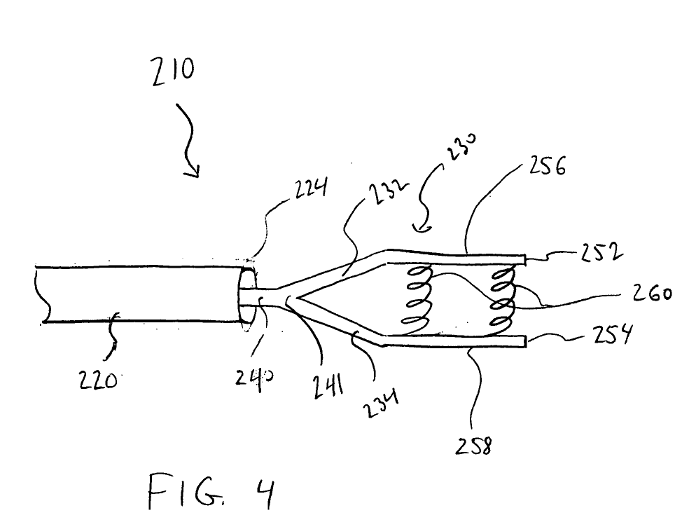

Referring now to FIG. 4, a perspective view of a system 210 according to another embodiment of the present invention is shown. As shown in FIG. 4, the system 210 comprises a cannula 220, an elongate member 240, a first plate 252, a second plate 254, and a plurality of springs 260 disposed between the first and second plates 252, 254. The first and second plates 252, 254 are configured to be slidably disposed within the cannula 220. The plurality of springs 260 are configured to be at least partly compressed when the first and second plates 252, 254 are within the cannula 220. The plurality of springs 260 are further configured to decompress, at least in part, as the first and second plates 252, 254 are extended beyond a distal end 224 of the cannula 220.

Disposed at a distal end 241 of the elongate member 240 are a first deflectable beam 232 and a second deflectable beam 234. The first plate 252 is coupled to the first deflectable beam 232, and the second plate 254 is coupled to the second deflectable beam 234. The first plate 252 comprises a first surface 256 configured to contact a tissue. Similarly, the second plate 254 comprises a second surface 258 also configured to contact tissue.

In the illustrative embodiment shown in FIG. 4, the elongate member 240, the first and second deflectable beams 232, 234, and the first and second plates 252, 254 are all fashioned from a single piece of metal 230. The system 210 shown may be used to provide a new cavity or adjust an existing cavity in an interior body region. The piece of metal 230 can be formed using a variety of suitable techniques, including, for example, machining, die casting, forging, grinding, and injection molding.

In the embodiment shown, the single piece of metal 230 comprises a titanium alloy material comprising a shape memory properties. In other embodiments, one or more of the components of the system 210 may be fashioned from a different material or may be comprise a separate piece coupled to the other components of the system 210. Due to the shape memory properties of the single piece of metal 230, the first and second deflectable beams 232, 234 comprise a tendency to spring open to assume a preset, native expanded dimension between the first and second plates 252, 254 as shown in FIG. 4 once they have been extended beyond the distal end 224 of the cannula 220.

As described above, the plurality of springs 260 are also configured to decompress as the first and second plates 252, 254 are extended beyond the distal end 224 of the cannula 220. Accordingly, the plurality of springs 260 provide a force that may assist the single piece of metal 230 assume its preset, native expanded dimension between the first and second plates 252, 254 when body tissues in contact with the first and second surfaces 256, 258 provide a resistive force that the shape memory properties of the single piece of metal 230 cannot overcome. As such, a user of the system 210 may be able to provide force to surrounding tissues to provide a cavity of desired shape and dimension within a treatment area.

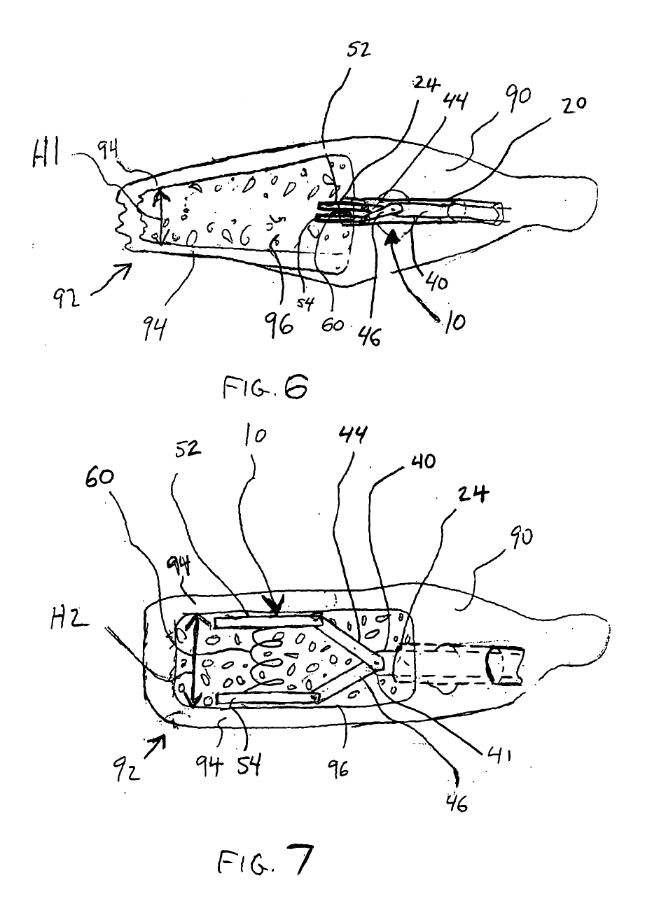

Upon provision of such a cavity, the first and second plates 252, 254 and the plurality of springs 260 may then be removed from the interior body region through the cannula 220. Once removed, a material, such as a bone cement, may then be used to fill a cavity provided by the system 210. Such an embodiment may be useful in situations where the system 210 is used to restore height to a vertebral body (see FIGS. 6-7). The bone cement may be inserted, either via the cannula 220, or via a separate cannula (such as a contralateral cannula).

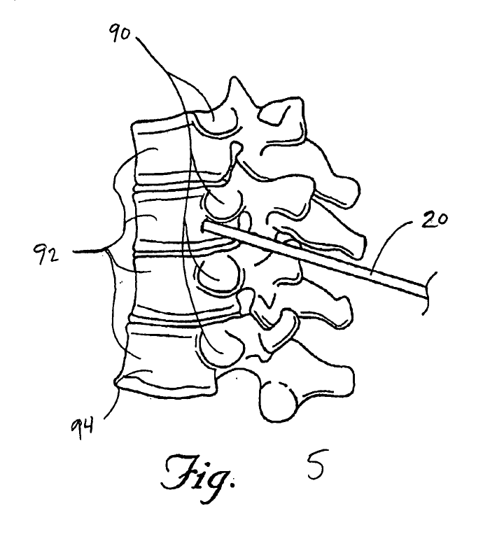

Referring now to FIG. 5, an elevation (lateral) view of several human vertebrae 90 is shown, with a cannula 20 establishing a percutaneous path along its axis to a vertebral body 92 of one of the several vertebrae. The vertebral body 92 extends on the anterior (i.e., front or chest) side of the vertebra 90. The vertebral body 92 comprises an exterior formed from compact cortical bone 94. The cortical bone 94 encloses an interior volume of reticulated cancellous, or spongy, bone 96 (also called medullary bone or trabecular bone—shown in FIGS. 6-7).

The vertebral body 92 is in the shape of an oval disc. As FIGS. 5-7 show, access to the interior volume of the vertebral body 92 can be achieved, e.g., by drilling an access portal through a rear side of the vertebral body 92, (a postero-lateral approach). The portal for the postero-lateral approach enters at a posterior side of the vertebral body 92 and extends anteriorly into the vertebral body 92. The portal can be provided either with a closed, minimally invasive procedure or with an open procedure.

Alternatively, access into the interior volume can be accomplished by drilling an access portal through one or both pedicles of the vertebra 90. This is called a transpedicular approach. Access into the interior of the vertebral body may also be accomplished using an extrapedicular approach alongside a pedicle of the vertebra 90, or from the anterior side. It is the physician who ultimately decides which access site is indicated.

A tool according to the present invention may be configured to be deployed within or adjacent at least one layer of tissue by movement within and along a path formed by the axis of the cannula 20. For example, as shown in FIG. 5, the cannula 20 may be part of a system, such as the systems 10 or 210 described above, with access to the cancellous bone within the vertebral body 92 of a vertebra 90 to provide a cavity therewithin. Such a cavity may be provided during a procedure for restoring some of the height of a vertebral body lost due to a vertical compression fracture or other pathology or trauma, prior to insertion of a bone cement into the vertebral body 92.

It should be appreciated, however, that systems and methods according to the present invention are not limited in application to human vertebrae, and may be used to provide cavities within or curette other parts of a living or non-living organism. For example, the system 10 can be deployed in other embodiments in other bone types and within or adjacent other tissue types, such as in a vertebral disc a knee joint, etc.

Referring now to FIG. 6, an elevation (lateral) view of a human vertebra 90 comprising a vertical compression fracture condition is shown. As shown in FIG. 6, a vertebral body 92 of the vertebra 90 has been partially crushed due to an osteoporotic condition of cancellous bone 96 therewithin. The dimension H1 of the vertebral body 92 has been decreased as a result of this fracture.

The system 10 as described above with respect to FIGS. 1-3 is shown in an unexpanded state (see FIG. 2). The spring 60, is shown compressed while the first and second plates 52, 54 are shown partly within the cannula 20. A user of the system 10 may wish to use it to provide a cavity within the vertebral body 92, and to restore height to the vertebral body 92 lost when the fracture occurred. The cannula 20 has been percutaneously inserted to provide access to the cancellous bone 96 within the vertebral body 92. The cannula 20 is shown with portions removed to reveal the elongate member 40, the coupling arms 44, 46, the plates 52, 54, and the spring 60 therewithin. The first and second plates 52, 54 are configured to move into the vertebral body 92 within and along the axis of the cannula 20.

In the embodiment shown in FIG. 6, while the first and second plates 52, 54 are at least partly within the cannula 20, the spring 60 is at least partly compressed. In this embodiment, once the first and second plates 52, 54 are fully extended beyond the distal end 24 of the cannula 20, the spring 60 decompresses, at least partially. The decompression of the spring 60 causes the distance between the first and second plates 52, 54 to increase, thereby increasing a dimension of a cavity within the cancellous bone 96.

In use, the elongate member 40 is substantially carried for sliding within the cannula 20. The user of the system 10 may freely slide the elongate member 40 axially within the cannula 20 to deploy the first and second plates 52, 54 and the spring 60 in a targeted treatment site. When deployed at the site, the user can extend the first and second plates 52, 54 beyond the distal end 24 of the cannula 20 adjacent cancellous bone tissue 96 within the vertebral body 92. In some embodiments the user may also able to rotate the elongate member 40 within the cannula 20 and thereby the first and second plates 52, 54 to adjust at least one of their orientation and travel path.

Referring now to FIG. 7, an elevation (lateral) view of the human vertebra 90 of FIG. 7 is shown after the system 10 has increased the height of the vertebral body 92 to dimension H2 from dimension H1 as shown in FIG. 6. As shown in FIG. 7, the first and second plates 52, 54 coupled to the distal end 41 of the elongate member 40 have been fully extended beyond the distal end 24 of the cannula 20. The spring 60 has decompressed, providing a force configured to increase the dimension between the first and second plates 52, 54. Resultantly, the first and second plates 52, 54 are shown in a fully expanded state (as shown in FIG. 1). Such an increase in the dimension H2 may allow a physician using the system 10 to at least partially restore the vertebra 90 to a shape analogous to its pre-vertical compression fracture condition.

In one embodiment, a suction tube may also be deployed through the cannula 20 to remove cancellous bone fragments dislodged by the system 10. In yet another embodiment, the system may comprise an interior lumen to serve as a suction tube as well as to convey a rinsing liquid into the cavity as it is being formed. The suction tube (or a lumen) may introduce a rinsing fluid (with an anticoagulant, if desired) and may remove cancellous bone dislodged by the system 10. Alternatively, the cannula 20 may comprise a first interior lumen that serves as a suction tube, and a second interior lumen that serves to flush the treatment area.

Once the desired cavity C is formed, the cavity-providing tool, such as system 10, may be withdrawn through the cannula 20. In one embodiment, the cavity C may then be at least partially filled with a material, such as a bone cement. Any other suitable tool can then be deployed through the cannula 20, or through another cannula (such as a contralateral cannula) into the formed cavity C. A second tool can, for example, perform a diagnostic or therapeutic procedure (such as filling the cavity C with a bone cement). In other embodiments other materials (such as a therapeutic material) may be provided into the cavity C by at least one of the first plate 52, the second plate 54, the first coupling arm 44, the second coupling arm 46, the spring 60, and the elongate member 40 while they are deployed in the vertebral body 92.

For example, an allograft material, a synthetic bone substitute, a medication, or a flowable material that may set to a hardened condition may be provided into the cavity C. The procedure may also be used to apply radiation therapy or chemotherapy. Further details of the injection of such materials into the cavity C for therapeutic purposes may be found in U.S. Pat. Nos. 4,969,888 and 5,108,404, and in co-pending U.S. patent application Publication No. 2003/0229372, which are incorporated herein by reference.

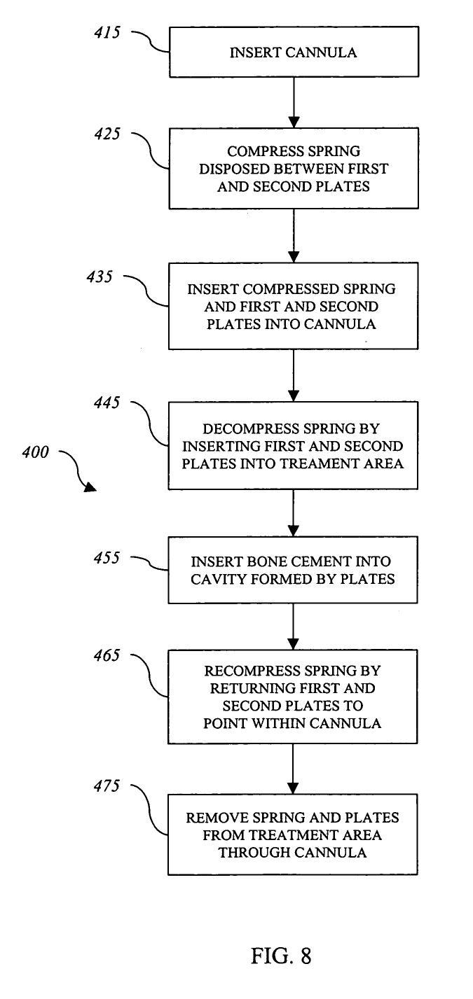

Referring now to FIG. 8, a flow chart of a method 400 according to one embodiment of the present invention is shown. The illustrative embodiment comprises percutaneously inserting a cannula (such as the cannula 20 described above) into a vertebral body of a vertebra comprising a vertical compression fracture condition as shown in box 415. For example, the cannula may be inserted into the vertebral body as shown in FIGS. 5-7. In another embodiment a cannula may be inserted into another interior body region.

The method 400 further comprises compressing a spring disposed between a first plate and a second plate, as shown in box 425. The spring may comprise, for example, the spring 60 described above. The first and second plates may comprise, for example, the first and second plates 52, 54 described above. In one embodiment, the spring may be compressed manually when a user presses together the first and second plates. In another embodiment, a machine may be configured to automatically compress the spring by a predetermined amount.

The method 400 further comprises inserting the compressed spring and the first and second plates into the cannula, as shown in box 435. In one embodiment, the first and second plates may be coupled to the distal end of an elongate member. In such an embodiment, a user may manually insert the compressed spring and the first and second plates into the cannula. In another embodiment the spring may be inserted into the cannula by a machine. In one such embodiment, the first plate, the second plate, and the spring may come prepackaged within the cannula for use in an interior body region. In another embodiment, the spring may be compressed as a result of its insertion into the cannula with the first and second plates. For example, the cannula's proximal end may comprise a larger interior bore dimension than its distal end, allowing the spring and plates to enter the proximal end uncompressed, but compressing the spring as the plates are pushed toward the distal end.

The method 400 further comprises decompressing the spring by inserting the first and second plates into a treatment area located beyond the distal end of the cannula, as shown in box 445. The decompression of the spring increases a distance between the first and second plates, increasing a dimension in the treatment area. In one embodiment, the spring may be decompressed until a distance between the first and second plates comprises a predetermined dimension. For example, in the embodiment described with respect to FIG. 8, the treatment area is located within the vertebral body of the vertebra. The treatment area may comprise a cavity that has already been provided by another tool within the vertebral body. Such a treatment area may need to be enlarged or otherwise adjusted with the spring and plates to restore the vertebra to a dimension existing prior to a vertical compression fracture.

While compressed, the spring provides a force that tends to push the first and second plates apart. However, while within the cannula, the force provided by the spring is opposed by the inner wall of the cannula. Once beyond the distal end of the cannula, the force provided by the spring is opposed by tissue adjacent a first surface on the first plate and adjacent a second surface on the second plate. The opposing force provided by the tissue may be lesser than the opposing force provided by the inner wall of the cannula. In one embodiment, the spring may be decompressed until the first and second plates are substantially parallel.

The spring may comprise a spring constant such that the inner wall of the cannula prevents the spring from decompressing, but the spring may expand once beyond the distal end of the cannula in the treatment area. The spring may be selected based, at least in part, on the amount of force required to displace, fracture, or move the adjacent tissue in the treatment area.

The method 400 further comprises inserting a bone cement into the cavity formed, enlarged, or otherwise modified by the spring and plates, as shown in box 455. The bone cement may be inserted through the same cannula through which the spring and plates were inserted, or in another embodiment may be inserted through a separate cannula into the vertebral body. A separate cannula may be oriented in a contralateral manner to the cannula through which the spring and plates were inserted. The bone cement, which remains in the cavity, may provide dimensional stability to the vertebral body after the spring and plates have been removed.

Another surgical tool, such as a scope, may also be inserted into the cavity through the cannula. In a different embodiment, the user may elect not to insert the bone cement into the cavity, or may alternatively or additionally introduce a therapeutic material to the tissue in the treatment area. For example, in one embodiment, at least one of the first plate, the second plate, and the spring may have a therapeutic material applied to it prior to insertion into the treatment area beyond the distal end of the cannula.

The method 400 further comprises recompressing the spring by returning the first and second plates to a point within the distal end of the cannula, as shown in box 465. In one embodiment, the first and second plates may be coupled to an elongated member via first and second coupling arms, respectively. For example, the first and second coupling arms may comprise the first and second coupling arms 44, 46 described above. In such an embodiment, the coupling arms may come into contact with the distal end or interior surface of the cannula as at least one of the first and second plates are withdrawn from a treatment area beyond the distal end of the cannula to a point within the cannula. Contact between the coupling arms and either the distal end or the inner surface of the cannula may provide a force to the first and second plates, compressing the spring disposed therebetween.

In a different embodiment, a user may use a controller in communication with the spring or the plates. Such a controller may be able to adjust the amount the spring is compressed independent of the distance the spring extends or does not extend beyond the distal end of the cannula.

The illustrative method 400 finally comprises removing the recompressed spring and the first and second plates from the treatment area through the cannula, as shown in box 475. In one embodiment, the spring and plates may be removed from the cavity once a user has determined that an appropriate amount of height has been restored to a vertebral body suffering from a vertical compression fracture condition, or that a cavity of sufficient size and shape has been provided within the vertebral body. In a different embodiment, a bone cement or a therapeutic material may be introduced to the cavity in the treatment area after the spring and plates have been removed.

In other embodiments, at least one of the plates or the spring may be implanted within the treatment area, either with or without inserting the bone cement or another substance into the treatment area. For example, in one such embodiment, one or both of the plates may be separable from an elongated member used to insert them into the treatment area through the cannula. A spring according to such an embodiment may be left implanted in either a compressed or an uncompressed state within the treatment area while the elongated member is removed through the cannula.

A spring used by the illustrative method 400 or another embodiment of the present invention may be selected based, at least in part, on its spring constant and overall size. In one embodiment, a spring to be disposed between the first and second plates may be selected comprising a spring configured to provide enough force to increase a dimension in a treatment area, but not so stiff as to prevent a user from recompressing the spring by withdrawing the first and second plates into the cannula.

In one method according to an embodiment of the present invention, at least one of the first and second plates may comprise a sharp surface configured to directly contact and shear tissue in the treatment area. Such a method may comprise contacting the tissue in the treatment area with the sharp surface, thereby curetting tissue.

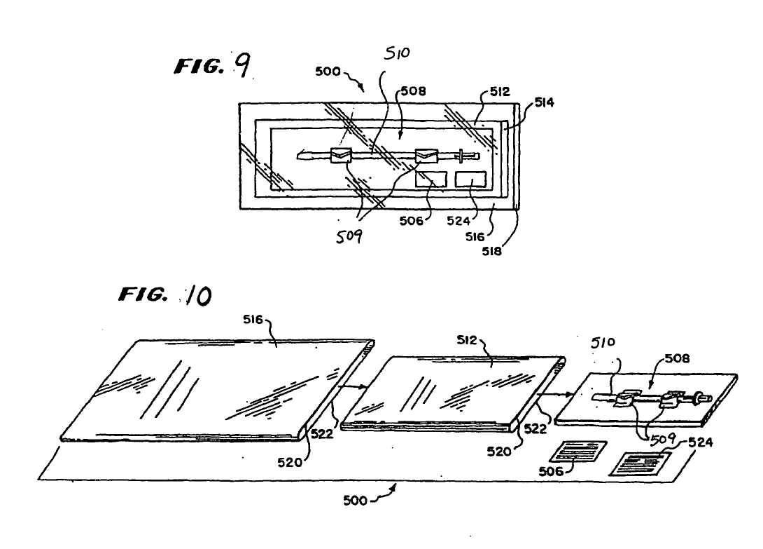

Referring now to FIGS. 9 and 10, a plan view and an exploded perspective view, respectively, of a sterile kit to store a cavity-forming tool according to one embodiment of the present invention is shown. A tool according to one embodiment of the present invention (such as the system 210 described above) may be packaged in a sterile kit 500 as shown in FIGS. 9 and 10 prior to deployment in a bone or other tissue. In one such embodiment, the tool may comprise a single use tool.

As shown in FIGS. 9 and 10, the kit 500 comprises an interior tray 508. The tray 508 holds the particular cavity-forming tool (generically designated 510) in a lay-flat, straightened condition during sterilization and storage prior to its first use. The tray 508 can be formed from die cut cardboard or thermoformed plastic material. The tray 508 comprises one or more spaced apart tabs 509, which hold the tool 510 in the desired lay-flat, straightened condition.

The kit 500 comprises an inner wrap 512 that, in the embodiment shown, is peripherally sealed by heat or the like, to enclose the tray 508 from contact with the outside environment. One end of the inner wrap 512 comprises a conventional peal-away seal 514 (see FIG. 10), to provide quick access to the tray 508 upon use, which may occur in a sterile environment, such as within an operating room.

The kit 500 shown also comprises an outer wrap 516, which is also peripherally sealed by heat or the like, to enclose the inner wrap 512. One end of the outer wrap 516 comprises a conventional peal-away seal 518 (see FIG. 10), to provide access to the inner wrap 512, which can be removed from the outer wrap 516 in anticipation of imminent use of the tool 510, without compromising sterility of the tool 510 itself.

Both inner and outer wraps 512 and 516 (see FIG. 10) comprise a peripherally sealed top sheet 520 and bottom sheet 522. In the illustrated embodiment, the top sheet 520 is made of transparent plastic film, like polyethylene or MYLAR™ material, to allow visual identification of the contents of the kit 500. The bottom sheet 522 may be made from a material permeable to ethylene oxide sterilization gas, e.g., TYVEC™ plastic material (available from DuPont).

The sterile kit 500 also carries a label or insert 506, which comprises the statement “For Single Patient Use Only” (or comparable language) to affirmatively caution against reuse of the contents of the kit 500. The label 506 also may affirmatively instruct against resterilization of the tool 510. The label 506 also may instruct the physician or user to dispose of the tool 510 and the entire contents of the kit 500 upon use in accordance with applicable biological waste procedures. The presence of the tool 510 packaged in the kit 500 verifies to the physician or user that the tool 510 is sterile and has not been subjected to prior use. The physician or user is thereby assured that the tool 510 meets established performance and sterility specifications, and will have the desired configuration when expanded for use.

The kit 500 also may comprise directions for use 524, which instruct the physician regarding the use of the tool 510 for creating a cavity in cancellous bone in the manners previously described. For example, the directions 524 instruct the physician to deploy, manipulate, and adjust the tool 510 inside bone to provide a cavity. The directions 524 can also instruct the physician to fill the cavity with a material, e.g., bone cement, allograft material, synthetic bone substitute, a medication, or a flowable material that sets to a hardened condition before, during, or after the tool 510 has provided the cavity.

The foregoing description of embodiments of the invention has been presented only for the purpose of illustration and description and is not intended to be exhaustive or to limit the invention to the precise forms disclosed. Numerous modifications and adaptations thereof will be apparent to those skilled in the art without departing from the spirit and scope of the present invention.

What is claimed is:

1. A system comprising:
a cannula comprising a cannula distal end;
an elongate member comprising an elongate member distal end;
a first plate configured to be slidably disposed within the cannula and coupled to the elongate member distal end;
a second plate configured to be slidably disposed within the cannula and coupled to the elongate member distal end; and
a spring disposed between the first and second plates, the spring configured to be at least partly compressed when the first and second plates are within the cannula, and to decompress, at least in part, as at least one of the first and second plates is extended beyond the cannula distal end, thereby increasing a distance between the first and second plates.

2. The system of claim 1, wherein the first and second plates are substantially parallel when the spring is at least partly decompressed.

3. The system of claim 1, wherein the first plate comprises a first surface facing a first direction, and the second plate comprises a second surface facing a second direction, wherein the first and second directions are separated by 180 degrees when the spring is at least partly decompressed, and the first and second surfaces are configured to be deployed in a treatment area adjacent a tissue.

4. The system of claim 1, wherein the spring is further configured to compress when at least one of the first and second plates is brought at least one of toward and within the cannula distal end.

5. The system of claim 1, wherein at least one of the first and second surfaces comprises a sharp surface configured to contact and shear a tissue.

6. The system of claim 1, further comprising:
a sheath circumscribing at least the first and second plates.

7. The system of claim 6, wherein the sheath comprises a substantially non-compliant material.

8. The system of claim 6, wherein the sheath comprises a rupture-resistant material.

9. The system of claim 6, wherein the sheath comprises an inflatable balloon.

10. The system of claim 1, wherein at least one of the first and second plates is configured to be implanted in a treatment area.

11. The system of claim 1, wherein at least one of the first plate, the second plate, the elongate member, and the spring comprises a shape memory material.

12. The system of claim 1, further comprising:
a first deflectable beam and a second deflectable beam are disposed at the elongate member distal end, wherein the first deflectable beam is coupled to the first plate, and the second deflectable beam is coupled to the second plate.

13. The system of claim 1, further comprising:
an actuator configured to at least one of increase and decrease the distance between the first and second surfaces.

14. A method comprising:
compressing a spring disposed between a first plate and a second plate;
inserting the compressed spring into a cannula comprising a cannula distal end; and
decompressing, at least in part, the spring once at least one of the first and second plates is inserted beyond the cannula distal end to a treatment area adjacent a tissue, thereby increasing a distance between the first and second plates.

15. The method of claim 14, wherein decompressing the spring comprises decompressing the spring until the first and second plates are substantially parallel.

16. The method of claim 14, wherein decompressing the spring comprises decompressing the spring until the distance between the first and second plates comprises a predetermined dimension.

17. The method of claim 14, further comprising:
recompressing, at least in part, the spring; and
removing the recompressed spring through the cannula.

18. The method of claim 17, wherein recompressing the spring comprises removing at least one of the first and second plates from the treatment area to a point within the cannula distal end.

19. The method of claim 14, wherein at least one of the first and second surfaces comprises a sharp surface configured to directly contact and shear the tissue, and further comprising:
contacting the tissue with the sharp surface.

20. The method of claim 14, further comprising:
inserting a bone cement into the treatment area.

21. The method of claim 14, further comprising:
applying a therapeutic material to at least one of the first plate, the second plate, and the spring.

22. The method of claim 21, further comprising:
introducing the therapeutic material to the tissue.

23. The method of claim 14, further comprising:
implanting at least one of the first and second plates.
